(19) United States
(12) Patent Application Publication
(10) Pub. No.: US 2026/0207424 A1
(43) Pub. Date: Jul. 23, 2026

(54) TRAVEL MASSAGER

(71) Applicant: Keith D. Cheatham, II, Girard, OH (US)

(72) Inventor: Keith D. Cheatham, II, Girard, OH (US)

(21) Appl. No.: 19/181,460

(22) Filed: Apr. 17, 2025

Related U.S. Application Data
(60) Provisional application No. 63/639,360, filed on Apr. 26, 2024.

(51) Int. Cl. A61H 23/02; A61H 9/00; A61H 15/00; A61H 39/04; A61N 1/36

(52) U.S. Cl. CPC A61H 23/0254; A61H 9/0092; A61H 15/0078; A61H 39/04; A61N 1/3603; A61H 2015/0021; A61H 2201/0134; A61H 2201/0149; A61H 2201/0157; A61H 2201/0161; A61H 2201/0207; A61H 2201/0214; A61H 2201/10; A61H 2201/164; A61H 2201/5002; A61H 2201/5005; A61H 2201/5097; A61H 2205/106

(57) ABSTRACT

A multi-use portable travel massager case comprising a massager cushion with integrated massage elements. The case includes a remote control for operating the massage elements and adjusting settings. At least one calf massager is included, providing targeted massage to the user's calves. The case features a foldable design with a storage compartment for accessories. Optional features include heating and cooling elements, wireless functionality, and integrated controls on the case. Various power adapter options are available. The invention incorporates a non-slip feature and may include a plastic protector for internal components. The case is constructed of water-resistant materials. Alternate designs offer different storage methods for calf massagers and personal items. The remote control enables independent or simultaneous operation of the massager cushion and calf massagers, providing a versatile, portable solution for customizable massage experiences in various settings.

### CROSS REFERENCE TO RELATED APPLICATIONS

This application claims the benefit of U.S. Provisional Patent Application 63/639,360, filed on Apr. 26, 2024, and incorporated herein by reference.

### STATEMENT REGARDING FEDERALLY SPONSORED RESEARCH AND DEVELOPMENT

Not Applicable.

### FIELD OF THE INVENTION

The invention relates generally to a multi-use portable travel massager case that combines a massage cushion, calf massagers, and storage functionality in a single, compact device.

### BACKGROUND

The field of personal massage devices has seen considerable growth, with a variety of products available to consumers seeking therapeutic relief. These devices range from handheld massagers to larger, stationary units designed for home or professional use. Many of these products aim to provide relief for muscle tension, soreness, and general discomfort associated with daily activities or more strenuous physical exertion.

Portable massage devices have gained popularity due to their convenience and ability to provide on-the-go relief. These typically include compact, battery-operated units that can be easily carried in a bag or suitcase. Some designs incorporate multiple massage techniques, such as vibration, shiatsu, or percussion, to cater to different user preferences and needs.

Larger, more comprehensive massage systems often come in the form of massage chairs or cushions that can be placed on existing furniture. These devices usually offer a wider range of massage options and can target multiple areas of the body simultaneously. However, their size and weight generally limit their portability.

Despite the advancements in personal massage technology, current solutions have significant drawbacks. Many portable and lightweight massagers are designed to target only one area of the body at a time, which is insufficient for users who experience soreness in multiple areas simultaneously. Larger devices that can massage multiple body parts are often too bulky for convenient travel. Additionally, while some devices offer various types of therapeutic relief, they often require the user to select a single massage technique for a specific area, not accommodating the common need for different types of relief for multiple body parts at the same time. Given these limitations, there is a clear need for an invention that combines portability with the ability to massage multiple areas simultaneously, offers various massage techniques, and serves as a convenient carrying case for easy transport and storage. The present invention accomplishes these objectives.

### SUMMARY OF THE INVENTION

The present invention is a multi-use portable travel massager case comprising a massager cushion with integrated massage elements. The massager cushion is constructed of durable and flexible materials and includes vibration motors, shiatsu rollers, or a combination thereof, designed to provide therapeutic relief to various body parts.

A remote control is electrically connected to the massager cushion, allowing users to operate the massage elements and adjust settings such as intensity, speed, and massage type. The remote control is equipped with a display screen and intuitive buttons for ease of use.

The invention includes at least one calf massager that provides targeted massage to the user's calves and is electrically connected to the remote control. The calf massager may incorporate air compression, vibration, or shiatsu massage techniques.

The massager case features a foldable design with a crease, allowing for easy storage and transport. A storage compartment is integrated into the case to accommodate the calf massager, remote control, and other personal items.

The massager cushion may incorporate heating and cooling elements to enhance the massage experience. Wireless functionality is an optional feature, allowing for cordless operation of the massage elements and calf massager.

The invention includes various power adapter options and may incorporate a pulley mechanism to secure the device to chairs or other surfaces. A belt or buckle feature may be included to secure the case when folded or to attach it to chairs or car seats.

The massager case includes a non-slip feature on the bottom and may incorporate a plastic protector to safeguard the internal components. The case is constructed of water-resistant or waterproof materials to protect the internal components from moisture and spills.

The invention offers versatility in design, with alternate storage methods for the calf massagers and personal items. The remote control enables users to operate the massager cushion and calf massagers either independently or simultaneously, providing a customizable massage experience.

The present invention addresses the drawbacks of the prior art by providing a versatile, portable massage device that combines multiple functionalities in a compact, travel-friendly design. Unlike existing solutions that are limited to targeting single body areas or offering only one massage technique at a time, this invention allows for simultaneous massage of multiple body parts using various therapeutic methods. The device integrates a massager cushion with calf massagers, all controllable through a single remote, enabling users to customize their massage experience according to their specific needs. By incorporating the massage components into a carrying case, the invention solves the portability issues associated with larger massage systems while offering more comprehensive coverage than typical handheld devices. Additionally, the inclusion of storage compartments for personal items enhances its practicality for travel use.

This innovative design effectively addresses the need for a portable, multi-functional massage solution that can provide targeted relief to various body areas simultaneously, making it an ideal choice for users seeking on-the-go therapeutic massage options. Other features and advantages of the present invention will become apparent from the following more detailed description, taken in conjunction with the accompanying drawings, which illustrate, by way of example, the principles of the invention.

### DESCRIPTION OF THE DRAWINGS

FIG. 1 is a perspective view of the massager case showing the case in a closed position with hand openings for carrying;

FIG. 2 is a front close view of the massager case illustrating the arrangement of massage motors or shiatsu rollers within the case;

FIG. 3 is a side view of the massager case with an attached calf storage bag;

FIG. 4 is a perspective view of the massager case in an open configuration and showing the calf storage and the internal configuration of the case;

FIG. 5 is front view of the massager case in the open configuration, detailing the calf storage bag;

FIG. 6 is a front view of the massager case with massage components, calf cuffs, and a remote, including connection cords;

FIG. 7 is a perspective view of the massager case with calf cuffs and the remote, depicting the cords and massage components;

FIG. 8 is a similar perspective view to FIG. 7, showing the massager case, calf cuffs, remote, and power adapters for various power sources;

FIG. 9 is a side perspective view of the massager case in the open configuration, demonstrating how the calf storage detaches from the case;

FIG. 10 is a rear perspective view of the detached calf storage from the massager case, showing a fastening method;

FIG. 11 is a perspective view of the massager case and calf cuffs with the remote, illustrating the use of the device for foot and leg massage;

FIG. 12A is a top plan view of the remote, detailing its dimensions and connection ports;

FIG. 12B is a front elevational view of the remote of FIG. 12A;

FIG. 12C is a perspective view of the remote of FIG. 12A;

FIG. 13A is a side elevational view of the massager case, showing different storage options;

FIG. 13B is a bottom plan view of a non-slip feature on the case's bottom;

FIG. 14 is a perspective view of the calf massager in an open and ready-to-use position, including a cord attachment;

FIG. 15 is a perspective view of an alternate massager case in a closed position, featuring a zipper enclosure for calf massager storage;

FIG. 16 is a side perspective view of the alternate massager case in an open position, revealing the internal arrangement of massage motors and/or rollers and calf massager storage;

FIG. 17 is a front perspective view of FIG. 16, displaying the calf massagers stored within the case;

FIG. 18 is a perspective view of the complete alternate massage device, with the massager case and calf massager, including the remote and a vehicle power adapter;

FIG. 19 is a front view of a preferred embodiment of the massager case in an open position, showing the calf massagers enclosed in the storage bag;

FIG. 20 is an enlarged view of the calf massagers, detailing the exterior and potential interior massage mechanisms;

FIG. 21 is a perspective view of heating elements and a plastic protector of the invention;

FIG. 22 is a partial perspective view showing the connecting power cable;

FIG. 23 is a perspective view of a preferred embodiment showing rubber anti-slip pieces;

FIG. 24 is an alternate perspective view of the preferred embodiment displaying the calf massagers and the cords connected to the remote and massager case;

FIG. 25 is a perspective view of a complete preferred embodiment of the invention;

FIG. 26 is a perspective view of an alternate storage method for personal items with zipper closures;

FIG. 27 is a perspective view of an alternate fastening method by use of buttons;

FIG. 28 is a rear view of alternate storage method of the massager case with the calf massagers placed inside; and

FIG. 29 is an alternate rear view of another embodiment of the storage method with the calf massagers stored using a pouch method; and

FIG. 30 is a perspective view of a closed massager case, suitable for storing other items such as other types of massaging devices.

### DETAILED DESCRIPTION OF THE PREFERRED EMBODIMENT

Illustrative embodiments of the invention are described below. The following explanation provides specific details for a thorough understanding of and enabling description for these embodiments. One skilled in the art will understand that the invention may be practiced without such details. In other instances, well-known structures and functions have not been shown or described in detail to avoid unnecessarily obscuring the description of the embodiments.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively. Additionally, the words “herein,” “above,” “below” and words of similar import, when used in this application, shall refer to this application as a whole and not to any particular portions of this application. When the claims use the word “or” in reference to a list of two or more items, that word covers all of the following interpretations of the word: any of the items in the list, all of the items in the list and any combination of the items in the list. When the word “each” is used to refer to an element that was previously introduced as being at least one in number, the word “each” does not necessarily imply a plurality of the elements, but can also mean a singular element. When the words “substantially” or “about” are used, if a quantitative measurement is necessary, within 95% of “complete” or “exact” should be considered the meaning. The term “the invention” or “the present invention” should always be construed as “an embodiment of the invention.”

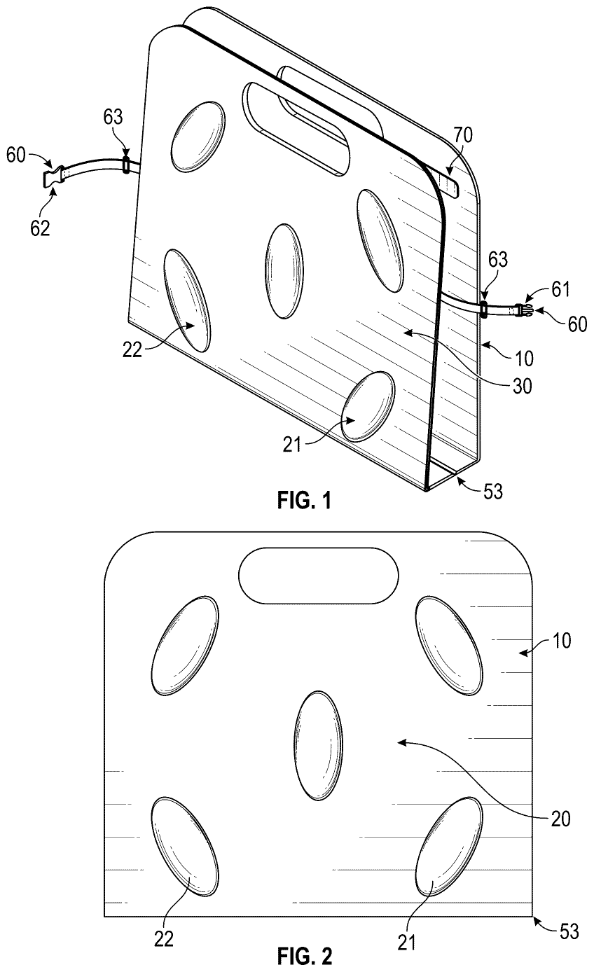

FIGS. 1 and 2 illustrate a multi-use portable travel massager case 10 comprising a massager cushion 20 with massage elements 21, 22. The massager cushion 20 is preferably constructed of durable and flexible materials such as neoprene, polyurethane, or the like. Massage elements 21, 22 are strategically arranged, preferably in a group of three to five, within the massager cushion 20 in a predetermined pattern to provide optimal coverage and therapeutic relief. These elements 21,22 may include vibration motors (not shown), which deliver rapid, pulsating movements to stimulate blood flow and relax muscles. Shiatsu roller elements 22 may also be incorporated, mimicking the pressing and kneading motions of traditional Japanese massage techniques. In some embodiments, a combination of both vibration motors and shiatsu rollers in the massage elements 21,22 may be used to offer a more diverse massage experience.

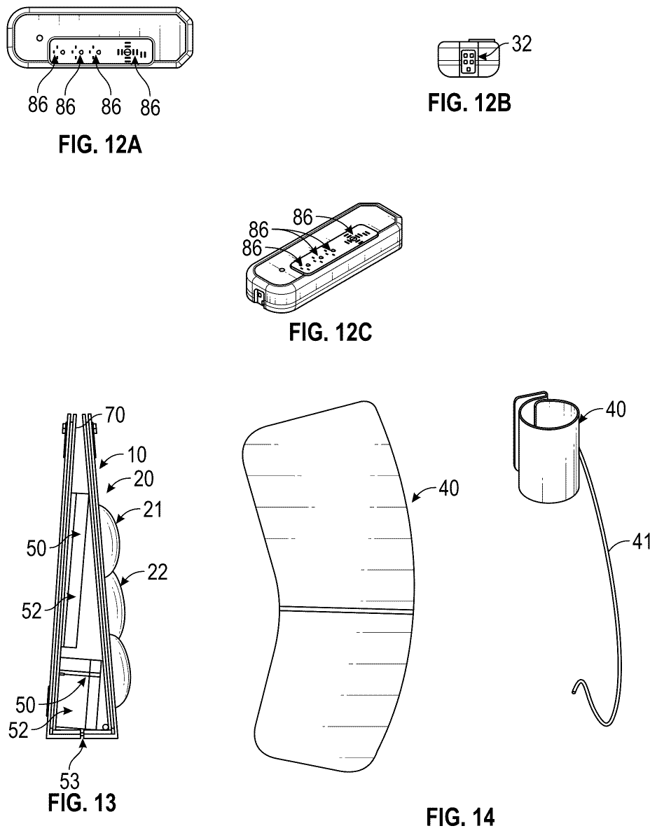

A remote control 30 is electrically connected to the massager cushion 20 via a cord 31, as shown in FIG. 6. The remote control 30 allows the user to operate the massage elements 21, 22 and adjust various settings such as intensity, speed, and massage type. Such a remote control 30 is preferably equipped with a display screen and intuitive buttons for ease of use, as detailed in FIGS. 12A, 12B, and 12C. The remote control 30 features at least one female plug connection 32, allowing the calf massagers 40 to be easily connected using a single insertion. The remote control 30 includes adjustable settings for massage intensity, speed, and massage type selection, including pulsating methods and heat therapy.

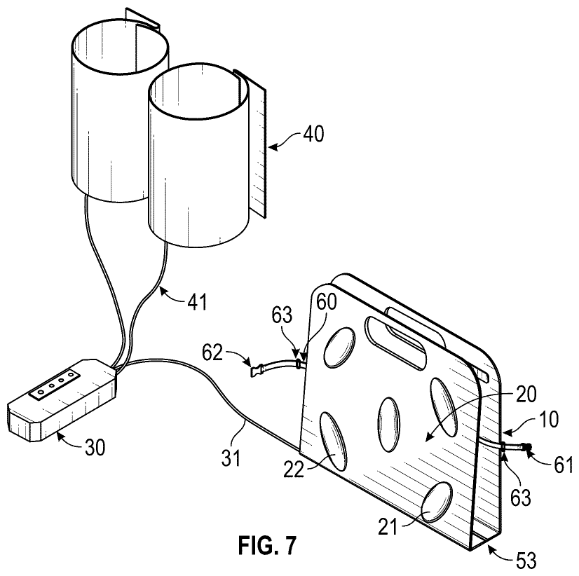

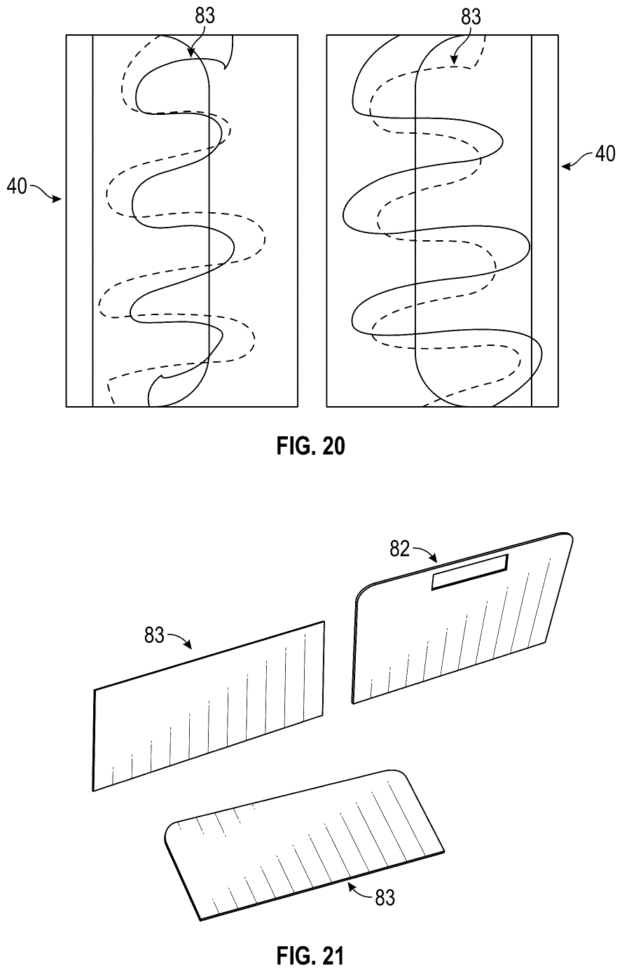

At least one calf massager 40 is included with the massager case 10, as illustrated in FIGS. 6 and 7. The calf massager 40 is designed to provide targeted massage to the user's calves and is electrically connected to the remote control 30 via a cord 41. Such a calf massager 40 may incorporate air compression, vibration, or shiatsu massage techniques. FIG. 20 provides an enlarged view of the calf massagers 40, detailing the exterior and potential interior massage mechanisms.

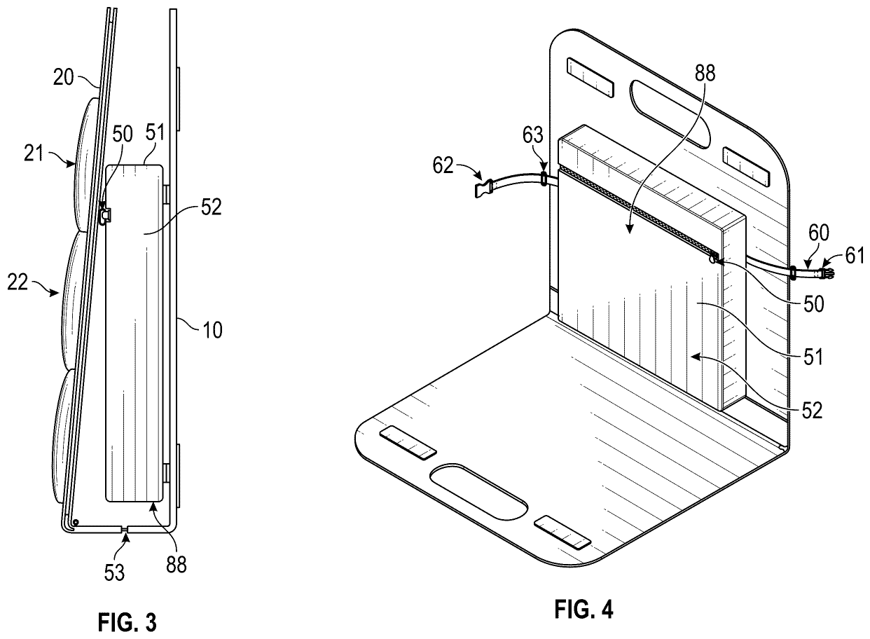

The massager case 10 features a foldable design with a crease 53, as shown in FIG. 3 and FIG. 4. This design allows for easy storage and transport of the device. The crease 53 is preferably reinforced with durable materials such as nylon webbing or heavy-duty stitching to ensure longevity. The foldable design allows the massager case 10 to close like a book's binding in a book-like manner, defining a top portion and a bottom portion, and enhancing its portability and ease of storage.

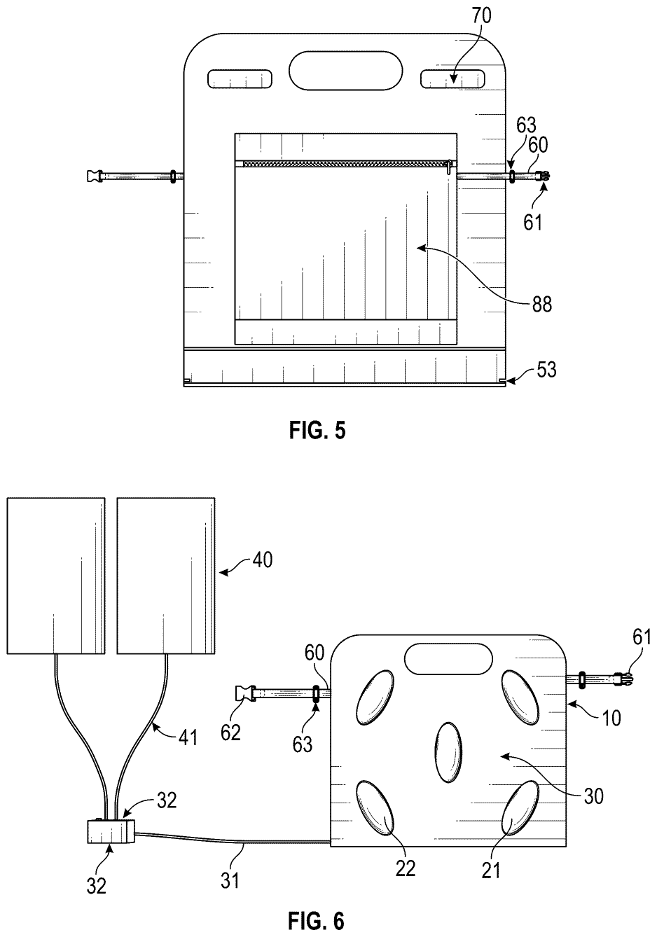

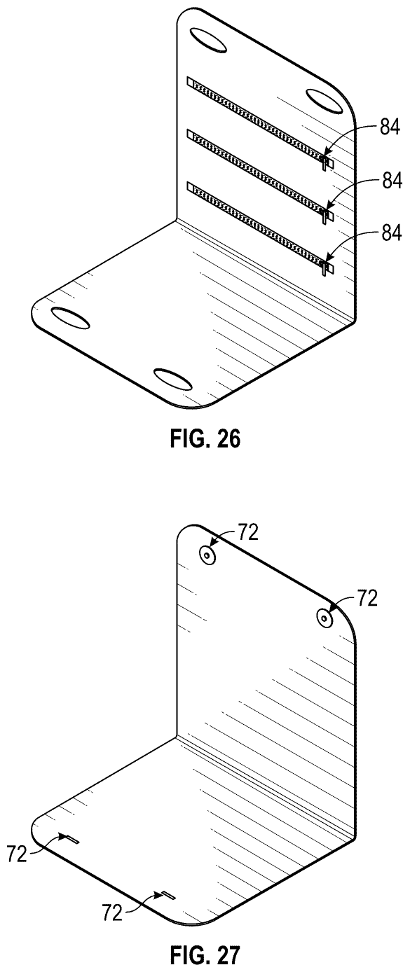

A storage compartment 50 is integrated into the massager case 10, as depicted in FIG. 4 and FIG. 5. Such a storage compartment 50 is designed to accommodate the calf massager 40, remote control 30, and other personal items. The storage compartment 50 may feature various pockets 51, dividers, or hollow regions 52 to organize contents effectively. FIG. 26 shows an alternate storage method for personal items with zipper closures 84.

The massager cushion 20 may incorporate heating and cooling elements 83 to enhance the massage experience. These elements are preferably controlled through the remote control 30 and can be adjusted to suit the user's preferences. FIG. 21 illustrates a perspective view of heating elements 83 and a plastic protector 82 of the invention.

Wireless functionality is an optional feature of the massager case 10. When implemented, it allows for cordless operation of the massage elements 21, 22 and calf massager 40, enhancing portability and convenience. The massager cushion 20 and the calf massagers 40 can be configured for wireless operation, each including a rechargeable battery for cordless functionality.

The massager case 10 may include integrated controls directly on the case, in addition to the remote control 30. Such integrated controls allow for quick adjustments without the need to locate the remote control 30.

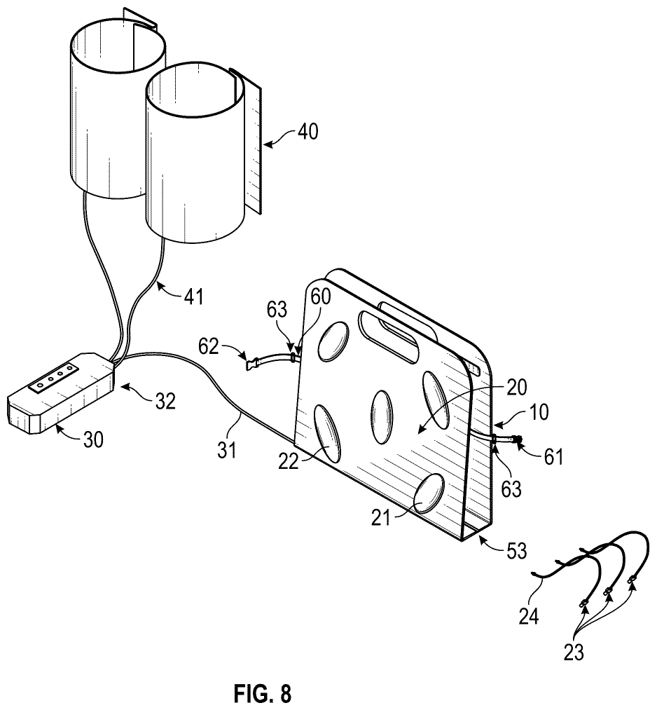

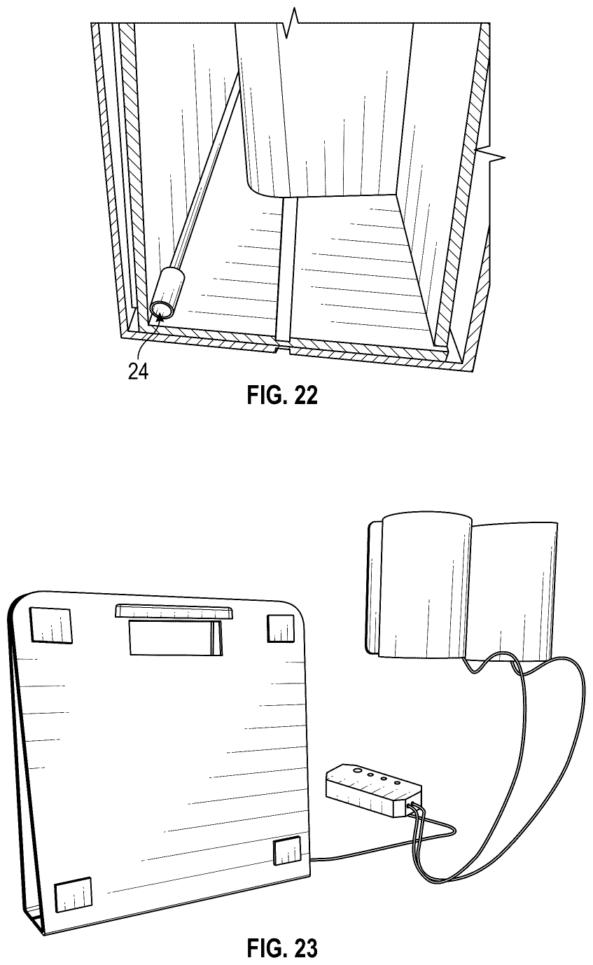

FIG. 8 illustrates various power adapter 23 options for the massager case 10. These may include a standard wall outlet home adapter, car adapter, and USB adapter, providing versatility in charging and powering the device. A connecting cable 24 links the power adapter 23 to the massager case 10, as shown in FIG. 22.

A pulley mechanism 63 may be incorporated into the massager case 10 to assist in securing the device to chairs or other surfaces. Such a pulley mechanism enhances the stability and positioning of the massager case 10 during use.

The calf massager 40 may be designed as a cordless unit with a rechargeable battery. This feature allows for greater flexibility in use and positioning of the calf massager 40. FIG. 14 shows a perspective view of the calf massager 40 in an open and ready-to-use position, including a cord 41 attachment.

FIG. 11 demonstrates how the massager case 10 can be used for foot and leg massage. The calf massager 40 is shown positioned for optimal therapeutic relief.

A belt or buckle feature 60 may be included to secure the massager case 10 when folded or to attach it to chairs or car seats. This feature includes male 61 and female 62 parts that clasp together, allowing the massager case 10 to be securely wrapped around seats and chairs. FIG. 27 illustrates an alternate fastening method using buttons 72.

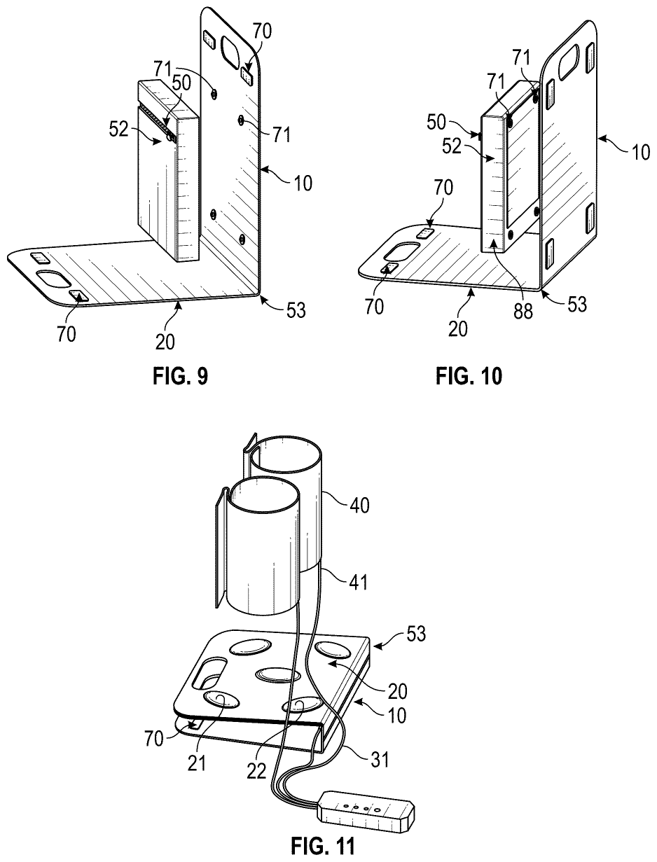

The massager case 10 may be secured using various fastening methods, including hook-and-loop type fasteners 70, snaps 71, or buttons 72, as shown in FIG. 27. These fastening methods ensure that the case remains closed during transport and storage. Additionally, snaps 71 or other fastening methods may be used to secure the removable storage bag 88 to the massager case 10 (FIGS. 9, 10 and 13A).

FIG. 13B illustrates a non-slip feature on the bottom of the massager case 10. This feature includes anti-slip pieces 64, as shown in FIG. 23, which help to keep the device in place during use on various surfaces.

A plastic protector 82 may be incorporated to safeguard the internal components of the massager cushion 20. Such a protector 82 enhances the durability and longevity of the device, as shown in FIG. 21.

The massager case 10 is preferably constructed of water-resistant or waterproof materials to protect the internal components from moisture and spills. Suitable materials may include treated fabrics, vinyl, or specialized waterproof textiles. Options include neoprene, rubber, Polyurethane Laminate (PUL), Thermoplastic Polyurethane (TPU), Extra-Long-Staple (ELS Cotton), Polyester and Nylon, Oilcloth, Microfiber, Wool, Latex, and Natural Rubber, or the like.

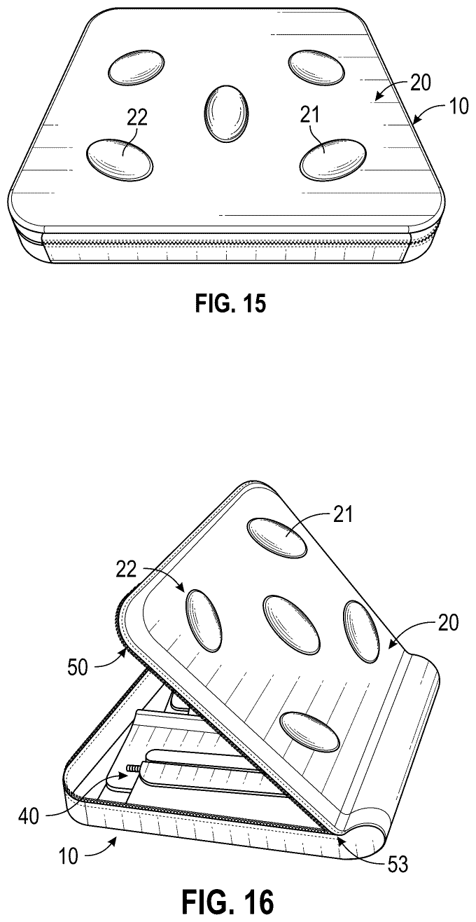

FIG. 15 shows an alternate design of the massager case 10 with a zipper enclosure for calf massager storage. This design provides secure storage and easy access to the calf massager 40.

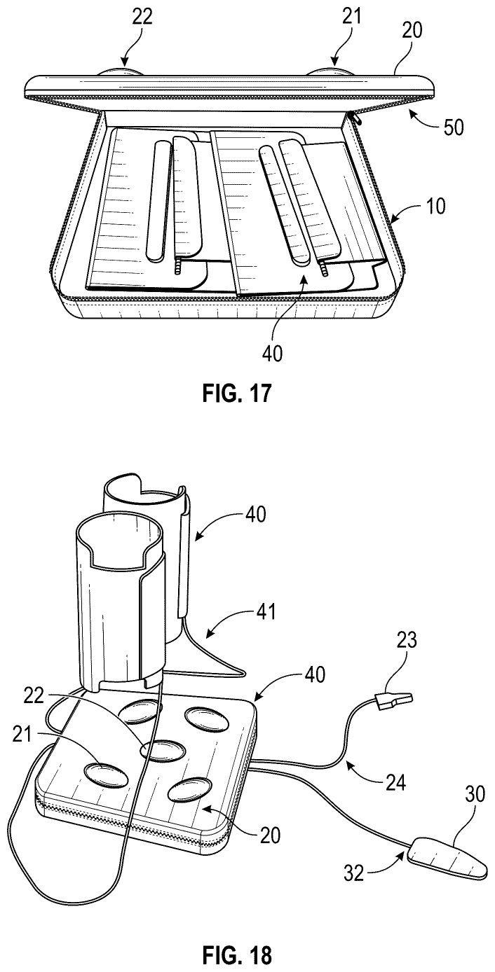

FIG. 16 and FIG. 17 illustrate an alternate massager case design with internal storage for the calf massagers 40. This configuration offers a more integrated and compact solution for storing all components of the device.

FIG. 18 demonstrates the complete massage device in use, including the massager case 10, calf massager 40, remote control 30, and vehicle power adapter 23. This figure highlights the versatility and portability of the device.

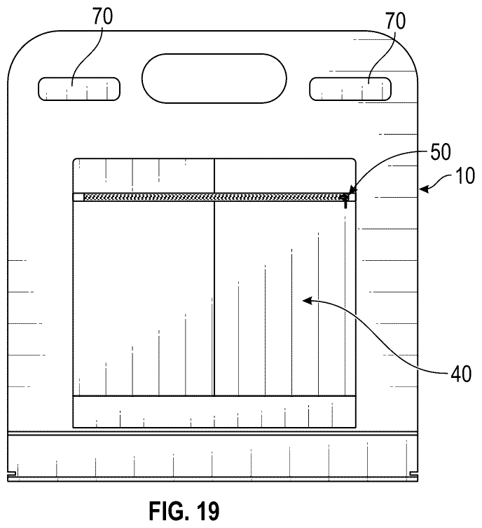

FIG. 19 shows a preferred embodiment of the massager case 10 in an open position, with the calf massagers 40 enclosed in a removable storage bag 88. This design offers protection for the calf massagers 40 when not in use and keeps all components organized within the massager case 10.

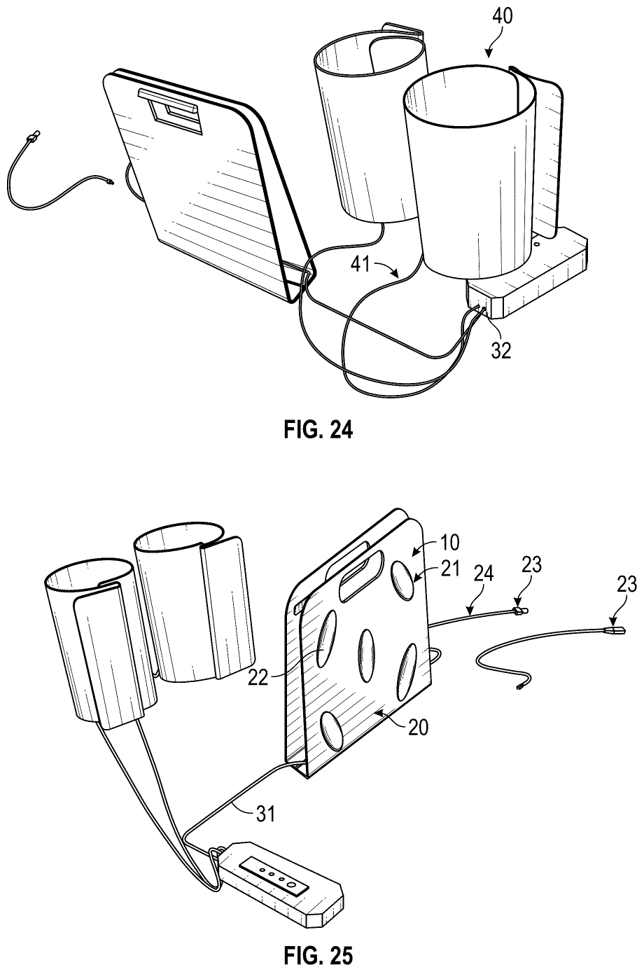

FIG. 24 provides an alternate perspective view of the preferred embodiment, displaying the calf massagers 40 and the cords 41 connected to the remote control 30 and massager case 10. FIG. 25 presents a perspective view of a complete preferred embodiment of the invention.

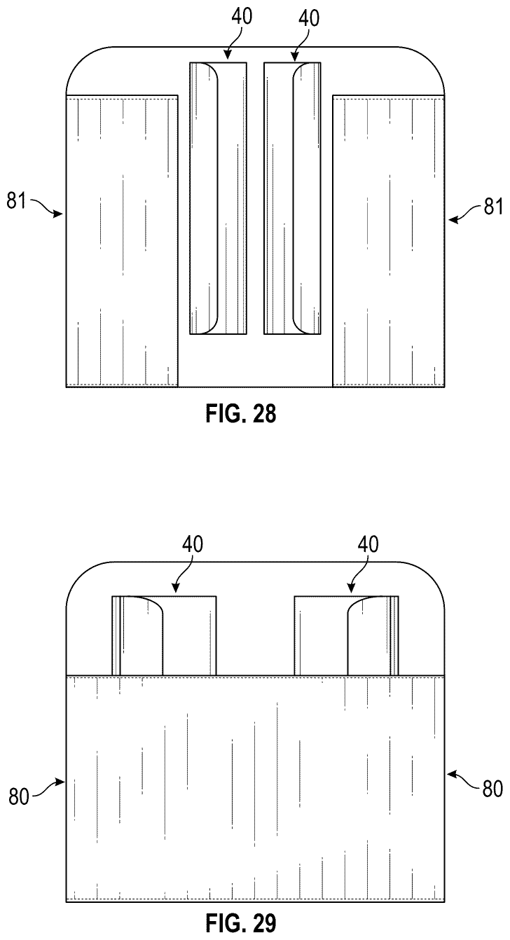

FIG. 28 illustrates a rear view of an alternate storage method of the massager case 10 with compartments 81 for storing the calf massagers 40. FIG. 29 shows an alternate rear view of another embodiment of the storage method with auxiliary storage pouches 80 for storing the calf massagers 40 using a pouch method. These auxiliary storage features on the back of the massager case 10 can also be used for storing personal items.

The massager cushion 20 is preferably approximately 15×15 inches in size, though this may vary to accommodate different user needs. In its basic version, the massager cushion 20 includes at least 3-5 motorized massage elements 21, providing comprehensive coverage for effective massage therapy.

The remote control 30 may offer various massage techniques, including vibration, shiatsu, and TENS unit therapy with electrical impulses. Such a remote control 30 allows users to adjust settings such as intensity, speed, pulsating methods, and heat.

The remote control 30 enables users to operate the massager cushion 20 and calf massagers 40 either independently or simultaneously, providing a customizable massage experience. This simultaneous operation allows for comprehensive therapeutic relief across multiple body areas.

A retractable securing mechanism (not shown), such as a belt, rope, or similar feature, may be included to facilitate the secure placement of the massager case 10 on various seating surfaces. This mechanism allows the user to easily wrap the device around chairs or seats, ensuring stability and optimal positioning during use.

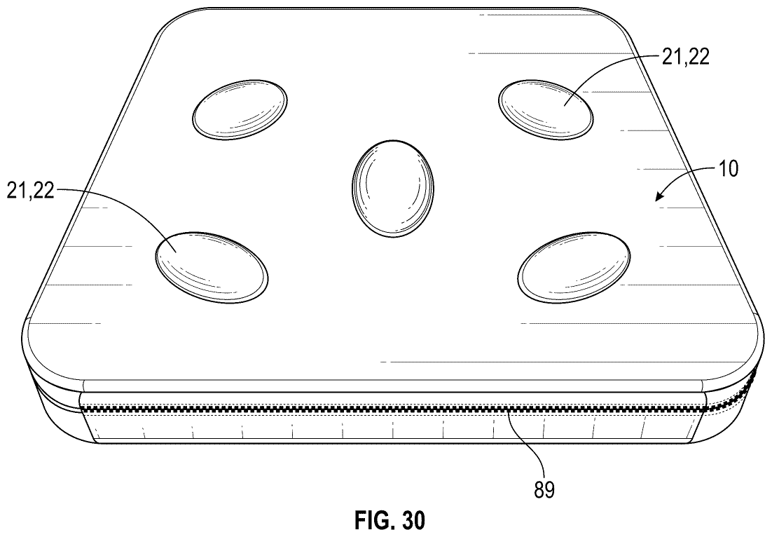

FIG. 30 illustrates a perspective view of the massager case 10 in a fully closed configuration. In this embodiment, the massager case 10 is secured by a zipper closure 89 that runs along the perimeter of the case, though other closure methods such as magnetic clasps, snap fasteners, or hook-and-loop fasteners could also be employed. When closed, the massager case 10 takes on a compact form factor, resembling a briefcase or travel bag. This design not only protects the internal massage components but also allows the case to serve as a versatile storage solution. The closed massager case 10 can accommodate various items beyond its primary massage function, such as additional massage devices, personal belongings, or travel essentials. This multi-functional aspect enhances the utility of the massager case 10, making it an ideal companion for travel or daily use, seamlessly transitioning between its roles as a massage device and a practical storage solution.

While a particular form of the invention has been illustrated and described, it will be apparent that various modifications can be made without departing from the spirit and scope of the invention. Accordingly, it is not intended that the invention be limited, except as by the appended claims.

Particular terminology used when describing certain features or aspects of the invention should not be taken to imply that the terminology is being redefined herein to be restricted to any specific characteristics, features, or aspects of the invention with which that terminology is associated. In general, the terms used in the following claims should not be construed to limit the invention to the specific embodiments disclosed in the specification, unless the above Detailed Description section explicitly defines such terms. Accordingly, the actual scope of the invention encompasses not only the disclosed embodiments, but also all equivalent ways of practicing or implementing the invention.

The above detailed description of the embodiments of the invention is not intended to be exhaustive or to limit the invention to the precise form disclosed above or to the particular field of usage mentioned in this disclosure. While specific embodiments of, and examples for, the invention are described above for illustrative purposes, various equivalent modifications are possible within the scope of the invention, as those skilled in the relevant art will recognize. Also, the teachings of the invention provided herein can be applied to other systems, not necessarily the system described above. The elements and acts of the various embodiments described above can be combined to provide further embodiments.

All of the above patents and applications and other references, including any that may be listed in accompanying filing papers, are incorporated herein by reference. Aspects of the invention can be modified, if necessary, to employ the systems, functions, and concepts of the various references described above to provide yet further embodiments of the invention.

Changes can be made to the invention in light of the above “Detailed Description.” While the above description details certain embodiments of the invention and describes the best mode contemplated, no matter how detailed the above appears in text, the invention can be practiced in many ways. Therefore, implementation details may vary considerably while still being encompassed by the invention disclosed herein. As noted above, particular terminology used when describing certain features or aspects of the invention should not be taken to imply that the terminology is being redefined herein to be restricted to any specific characteristics, features, or aspects of the invention with which that terminology is associated.

While certain aspects of the invention are presented below in certain claim forms, the inventor contemplates the various aspects of the invention in any number of claim forms. Accordingly, the inventor reserves the right to add additional claims after filing the application to pursue such additional claim forms for other aspects of the invention.

What is claimed is:

1. A multi-use portable travel massager case, comprising:
a massager cushion having a plurality of massage elements;
a remote control operatively connected to the massager cushion;
at least one calf massager operatively connected to the remote control;
wherein the massager cushion is configured to fold, having a crease connecting a top portion and a bottom portion;
a storage compartment integrated within the massager case for storing the at least one calf massager when not in use; and
wherein the remote control is configured to operate the massager cushion and the at least one calf massager independently or simultaneously.

2. The multi-use portable travel massager case of claim 1, wherein the plurality of massage elements comprises at least one of: vibration motors, shiatsu rollers, and TENS unit therapy elements.

3. The multi-use portable travel massager case of claim 1, further comprising heating and cooling elements integrated within the massager cushion and the at least one calf massager.

4. The multi-use portable travel massager case of claim 1, wherein the massager cushion and the at least one calf massager are configured for wireless operation.

5. The multi-use portable travel massager case of claim 1, further comprising integrated controls on the massager case for operating the massager cushion and the at least one calf massager.

6. The multi-use portable travel massager case of claim 1, wherein the plurality of massage elements comprise at least three to five massage motors arranged in a predetermined pattern across the massager cushion.

7. The multi-use portable travel massager case of claim 1, further comprising a pulley mechanism for adjusting a securing mechanism of the massager case.

8. The multi-use portable travel massager case of claim 1, wherein the at least one calf massager is cordless and includes a rechargeable battery.

9. The multi-use portable travel massager case of claim 1, wherein the massager cushion and the at least one calf massager are configured to provide at least two of: air compression massage, vibration therapy, shiatsu massage, and TENS unit therapy.

10. The multi-use portable travel massager case of claim 1, wherein the massager cushion is configured to fold along the crease in a book-like manner.

11. The multi-use portable travel massager case of claim 1, wherein the remote control includes adjustable settings for massage intensity, speed, and massage type selection.

12. The multi-use portable travel massager case of claim 11, wherein the massage type selection includes pulsating methods and heat therapy.

13. The multi-use portable travel massager case of claim 1, further comprising a belt or buckle feature with a male part and a female part for securing the massager case to a seat.

14. The multi-use portable travel massager case of claim 1, wherein the storage compartment comprises at least one of: a zippered pocket, a pouch, and a hollow region.

15. The multi-use portable travel massager case of claim 1, further comprising a plastic protector for safeguarding the plurality of massage elements.

16. The multi-use portable travel massager case of claim 1, wherein the remote control includes at least one female plug connection for connecting the at least one calf massager.

17. The multi-use portable travel massager case of claim 1, further comprising multiple power adapter options including at least two of: a home adapter, a car adapter, and a USB adapter.

18. The multi-use portable travel massager case of claim 1, wherein the massager case is constructed from a water-resistant or waterproof material.

19. The multi-use portable travel massager case of claim 1, further comprising auxiliary storage features on a back of the massager case for storing personal items.

20. The multi-use portable travel massager case of claim 1, wherein the massager cushion and the at least one calf massager are configured for wireless operation, each including a rechargeable battery for cordless functionality.
